(19) United States
(12) Patent Application Publication
(10) Pub. No.: US 2022/0118450 A1
(43) Pub. Date: Apr. 21, 2022

(54) MICROFLUIDIC DEVICES

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 17/417,500

(22) PCT Filed: Apr. 30, 2019
(86) PCT No.: PCT/US2019/029939

§ 371 Date: Jun. 23, 2021

(51) Int. Cl. B01L 3/00; B01L 7/00

(52) U.S. Cl. CPC B01L 3/502746; B01L 3/52; B01L 3/502761; B01L 7/00; B01L 3/502707; B01L 2200/12; B01L 2400/088; B01L 2300/16; B01L 2200/16; B01L 2300/18; B01L 2400/086

(57) ABSTRACT

The present disclosure relates to a microfluidic device including a microfluidic substrate and dry reagent-containing particles. The microfluidic substrate includes an ingress microfluidic channel that fluidly feeds an egress microfluidic channel through a microfluidic-retaining region that includes a microfluidic discontinuity feature, a particle-retaining chemical coating, or a combination thereof. The dry reagent-containing particles include a reagent that is releasable from the dry reagent-containing particles when exposed to a release fluid. The dry reagent-containing particles are retained within the microfluidic substrate at the microfluidic discontinuity feature or particle-retaining chemical coating in position to release the reagent into the egress microfluidic channel upon flow of release fluid from the ingress microfluidic channel through the microfluidic-retaining region.

### BACKGROUND

Microfluidic devices can exploit chemical and physical properties of fluids on a microscale. These devices can be used for research, medical, and forensic applications, to name a few, to evaluate or analyze fluids using very small quantities of sample and/or reagent to interact with the sample than would otherwise be used with full-scale analysis devices or systems.

### BRIEF DESCRIPTION OF THE DRAWINGS

FIG. 1 graphically illustrates a schematic view of an example microfluidic device in accordance with the present disclosure;

FIG. 2 graphically illustrates a schematic view of an example microfluidic device in accordance with the present disclosure;

FIG. 3 graphically illustrates a schematic view of an example microfluidic device in accordance with the present disclosure;

FIG. 4 graphically illustrates a schematic view of an example microfluidic device in accordance with the present disclosure;

FIG. 5 graphically illustrates a schematic view of an example microfluidic device in accordance with the present disclosure;

FIG. 6 graphically illustrates a schematic view of an example microfluidic device in accordance with the present disclosure;

FIG. 7 graphically illustrates a schematic view of an example microfluidic device in accordance with the present disclosure;

FIG. 8 graphically illustrates a schematic view of an example microfluidic device in accordance with the present disclosure;

FIG. 9 graphically illustrates a schematic view of an example dry reagent-containing particle in accordance with the present disclosure;

FIG. 10 graphically illustrates a schematic view of an example dry reagent-containing particle in accordance with the present disclosure;

FIG. 11 graphically illustrates a schematic view of an example dry reagent-containing particle in accordance with the present disclosure;

FIG. 12 graphically illustrates a schematic view of an example dry reagent-containing particle in accordance with the present disclosure;

FIG. 13 graphically illustrates a cross-sectional view of an example microfluidic system in accordance with examples of the present disclosure; and

FIG. 14 is a flow diagram illustrating an example method of manufacturing a microfluidic device in accordance with examples of the present disclosure.

### DETAILED DESCRIPTION

Microfluidic devices can permit the analysis of a fluid sample on the micro-scale. These devices utilize smaller volumes of a fluid sample and reagents during the analysis then would otherwise be used for a full scale analysis. In addition, microfluidic devices can also allow for parallel analysis thereby providing faster analysis of a fluid sample. For example, during sample analysis, a reagent can be delivered to interact with the sample fluid. A reagent can be used to removal chemicals that interfere with sensing and/or to aid in sensing. Introducing the reagent during sample analysis can increase the cost and skill associated with the analysis, the time associated with conducting sample analysis, and the potential for error. Further, some reagents can be susceptible to environmental degradation and/or can be hydrolyzed upon exposure to moisture, and some reagents that are not thermally stable can be degraded upon exposure to heat. As such, reagents that are protected from environmental degradation can provide benefits.

In accordance with an example of the present disclosure, a microfluidic device includes a microfluidic substrate and dry reagent-containing particles. The microfluidic substrate includes an ingress microfluidic channel that fluidly feeds an egress microfluidic channel through a microfluidic-retaining region. The microfluidic-retaining region includes a microfluidic discontinuity feature, a particle-retaining chemical coating, or a combination thereof. The dry reagent-containing particles include reagent that is releasable from the dry reagent-containing particles when exposed to release fluid. The dry reagent-containing particles are retained within the microfluidic substrate at the microfluidic discontinuity feature or particle-retaining chemical coating in position to release the reagent into the egress microfluidic channel upon flow of release fluid from the ingress microfluidic channel through the microfluidic-retaining region. In one example, the microfluidic discontinuity feature is present and includes a microfluidic cavity, a microfluidic weir, a microfluidic baleen, or a combination thereof. In another example, the microfluidic discontinuity feature is present and is associated with a porous membrane positioned downstream therefrom. The porous membrane having an average pore size to permit air, loading fluid, sample fluid, and released reagent in the presence of loading fluid to flow therethrough while prohibiting the dry reagent-containing particles from flowing therethrough while at the size of the particles after loading but before releasing reagent therefrom. In another example, the microfluidic discontinuity is present and includes a series of microfluidic cavities within the microfluidic-retaining region, wherein the series of microfluidic cavities are individually loaded with the dry reagent-containing particles. In yet another example, the series of microfluidic cavities are independently loaded with one of multiple different types of dry reagent-containing particles. In a further example, the microfluidic discontinuity feature is present and the particle-retaining chemical coating is also present. The particle-retaining chemical coating includes a streptavidin coating bound to a microfluidic channel wall surface of the microfluidic-retaining region. In one example, the microfluidic discontinuity feature is present and is a structural feature deviating from the ingress microfluidic channel and the egress microfluidic channel, and the structural feature includes the particle-retaining chemical coating present in the form of a streptavidin coating bound thereto. In another example, the microfluidic device further includes a thermal resistor associated with the microfluidic-retaining region and positioned to thermally interact with the dry reagent-containing particles to release reagent therefrom in the presence of a release fluid.

In another example, a microfluidic system includes a microfluidic substrate, dry reagent-containing particles, and a fluid carrier. The microfluidic substrate includes an ingress microfluidic channel that fluidly feeds an egress microfluidic channel through a microfluidic-retaining region, the microfluidic-retaining region including a microfluidic discontinuity feature, a particle-retaining chemical coating, or a combination thereof. The dry reagent-containing particles include reagent that is releasable from the dry reagent-containing particles when exposed to release fluid. The fluid carrier to combine or which is combined with the dry reagent-containing particles is inert with respect to the dry reagent-containing particles. In another example, the system further includes a loading apparatus to load the dry reagent-containing particles carried by the loading fluid to a location within the microfluidic-retaining region. In yet another example, when loading the dry reagent-containing particles with the loading apparatus, the microfluidic discontinuity or particle-retaining chemical coating is positioned to trap the dry reagent-containing particles at the microfluidic-retaining region.

Further presented is a method of manufacturing a microfluidic device. The method include loading a dry reagent-containing particle into a microfluidic-retaining region of a microfluidic substrate, the microfluidic substrate further including an ingress microfluidic channel that fluidly feeds an egress microfluidic channel through the microfluidic-retaining region, wherein the dry reagent-containing particles include a reagent that is releasable therefrom when exposed to a release fluid passed through the microfluidic-retaining region from the ingress microfluidic channel to the egress microfluidic channel. In one example, loading a dry reagent-containing particle into a microfluidic-retaining region includes loading a reagent into the micro-fluidic-retaining region and laminating the reagent to provide the dry reagent-containing particles within the microfluidic-retaining region. In another example, loading includes: passing a loading fluid through the microfluidic-retaining region which includes a fluid carrier and the dry reagent-containing particles, wherein the fluid carrier is inert with respect to the dry reagent-containing particles; and flowing a gas through the microfluidic channel to remove carrier fluid from the microfluidic channel while leaving dry reagent-containing particles at the microfluidic retaining region. In a further example, the method further includes passing a buffer solution through the microfluidic-retaining region prior to passing the loading fluid therethrough.

When discussing the microfluidic device, the microfluidic system, or the method of method of manufacturing a microfluidic device herein, such discussions can be considered applicable to one another whether or not they are explicitly discussed in the context of that example. Thus, for example, when discussing dry reagent-containing particles in the context of a microfluidic device, such disclosure is also relevant to and directly supported in the context of the microfluidic system and/or the method of manufacturing a microfluidic device, and vice versa.

Terms used herein will be interpreted as the ordinary meaning in the relevant technical field unless specified otherwise. In some instances, there are terms defined more specifically throughout or included at the end of the present disclosure, and thus, these terms are supplemented as having a meaning described herein.

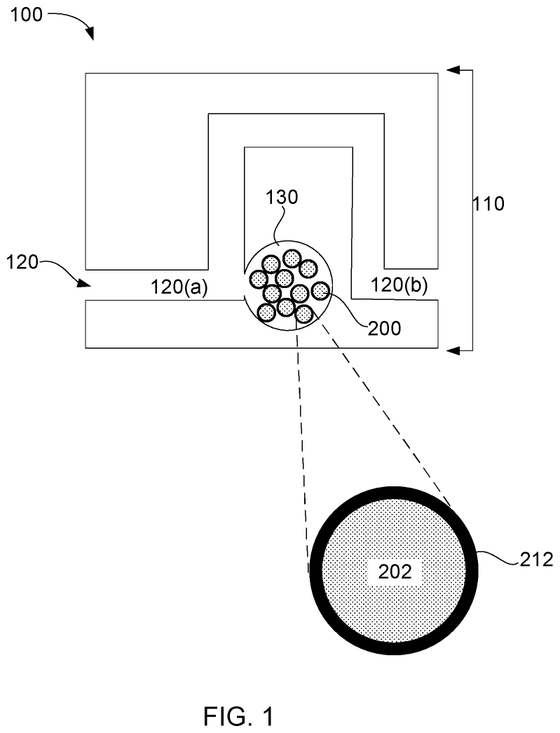

In accordance with the definitions and examples herein, FIGS. 1-8 and 13 depict various microfluidic devices. These various examples can include various features, with several features common from example to example. Thus, the reference numerals used to refer to features depicted in FIGS. 1-8 and 13 are the same throughout to avoid redundancy, even though the microfluidic devices and the microfluidic systems can have structural differences, as shown. Likewise, the reference to the dry reagent-containing particles includes the use of reference numerals where possible to also avoid redundancy in the present disclosure.

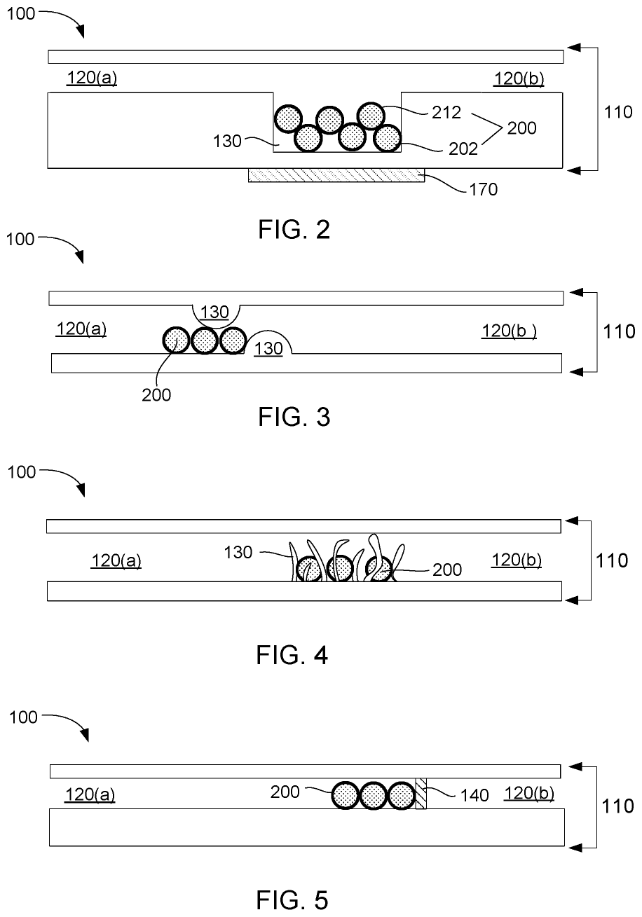

FIG. 1 depicts a schematic view of microfluidic device 100 that can include a microfluidic substrate 110 and a microfluidic-retaining region 130 that can be fluidly coupled to a microfluidic channel 120 (sometimes shown as 120(a) and 120(b) to show ingress opening and egress openings of the channel). Dry reagent-containing particles 200 can be positioned in the microfluidic-retaining region for release of a reagent 202 from a reagent carrier 212, such as a salt with reagent adsorbed thereon, a degradable polymer associated with the reagent, or some other particle that can release reagent upon exposure to a releasing fluid. However, as shown in FIG. 1, the reagent carrier can be a degradable polymer 212 that surrounds the reagent to be eroded to allow release of the reagent. Notably, FIGS. 2-9 depict similar features that are commonly indicated with the same reference numerals as shown in FIG. 1, with a notable difference in the various structures of the respective microfluidic-retaining regions shown in those FIGS. Thus, these FIGS. are described herein together to some extent.

The term “dry reagent-containing particles” does not indicate that the particles are dry at every point in time, such as during manufacture of the particles or loading of the particles in the microfluidic device, for example. To illustrate, dry reagent-containing particles can be loaded (dispersed) in a carrier fluid to form a loading fluid (to load the particles at the microfluidic discontinuity feature and/or particle-retaining chemical coating that retains the particles. The carrier fluid may be removed, leaving the dry reagent-containing particles (even if some moisture inherently remains). Thus, the dry reagent-containing particles can likewise be defined as particulates that can be loaded at a location within the microfluidic device or system, and from which reagent can be release when exposed to a release fluid.

Thus, in various examples herein, reagent 202 can be releasable from the reagent carrier 212, e.g. degradable polymer, when release fluid (not shown, as it would typically be present during use) is flowed through the microfluidic channel 120 and thus fluidly communicates with the microfluidic-retaining region 130. As used herein a “release fluid” can refer to a fluid that can degrade, dissolve, or erode the degradable polymer or can carry the reagent upon degradation, dissolution, or erosion of the degradable polymer by other means, such as UV light, heat, or enzymes.

The microfluidic substrate 110 can be a single layer or multi-layer substrate. The material of the microfluidic substrate can include glass, silicon, polydimethylsiloxane (PDMS), polystyrene, polycarbonate, polymethyl methacrylate, poly-ethylene glycol diacrylate, perflouroaloxy, fluorinated ethylenepropylene, polyfluoropolyether diol methacrylate, polyurethane, cyclic olefin polymer, teflon, copolymers, and combinations thereof. In one example, the microfluidic substrate can include a hydrogel, ceramic, thermoset polyester, thermoplastic polymer, or a combination thereof. In another example, the microfluidic substrate can include silicon. In yet another example, the microfluidic substrate can include a low-temperature co-fired ceramic.

The microfluidic channel 120 can be negative space that can be etched, molded, or engraved from the material of the microfluidic substrate or can be formed by wall of different sections of a multi-layer microfluidic substrate. The microfluidic channel can include an ingress microfluidic channel 120(a) and an egress microfluidic channel 120(b) and can have a channel size that can range from 1 μm to 1 mm in diameter. In yet other examples, the microfluidic channel can have a channel size that can range from 1 μm to 500 μm, from 100 m to 1 mm, from 250 μm to 750 μm, or from 300 μm to 900 μm, etc. The microfluidic channel can have a linear pathway, a curved path, a pathway with turns, a branched pathway, a serpentine pathway, or any other pathway configuration.

In one example, the microfluidic-retaining region 130 can include a microfluidic discontinuity feature. The microfluidic discontinuity feature can include a microfluidic cavity, microfluidic weir, microfluidic baleen, or a combination thereof. In one example, the microfluidic discontinuity feature can include a microfluidic cavity, such as that depicted schematically by example in FIGS. 1, 2, 7, and 8. In another example, the microfluidic discontinuity feature can include a microfluidic weir, such as that depicted by example in FIG. 3. In yet another example, the microfluidic discontinuity feature can include microfluidic baleen, such as that depicted schematically by example in FIG. 4. In some examples, the microfluidic discontinuity feature can include a combination of discontinuity features. The microfluidic discontinuity feature can be used to retain the dry reagent-containing polymer in the microfluidic-retaining region.

In some examples, as depicted in FIGS. 5 and 7 in particular, the microfluidic-retaining region 130 can be associated with a porous membrane 140. The porous membrane can be positioned downstream from the microfluidic-retaining region and can have an average pore size that can permit air, release fluid, sample fluid, and released reagent in the presence of a loading fluid to flow there through while prohibiting the dry reagent-containing particles 200 from flowing therethrough. The porous membrane can be operable to prevent migration of the dry reagent-containing particles after loading but before releasing reagent 202 therefrom. Accordingly, the porous membrane can have an average pore size that can be smaller than an average particle size of the dry reagent-containing particles but larger than the average particle size of the reagent. In some examples, the porous membrane can have an average pore size ranging from 5 μm to 70 μm, from 5 μm to 50 μm, from 7 μm to 70 μm, from 7 μm to 50 μm, from 12 μm to 70 μm, from 12 μm to 50 μm, from 15 μm to 50 μm, from 50 μm to 70 μm, from 5 μm to 25 μm, or from 25 μm to 60 μm, for example.

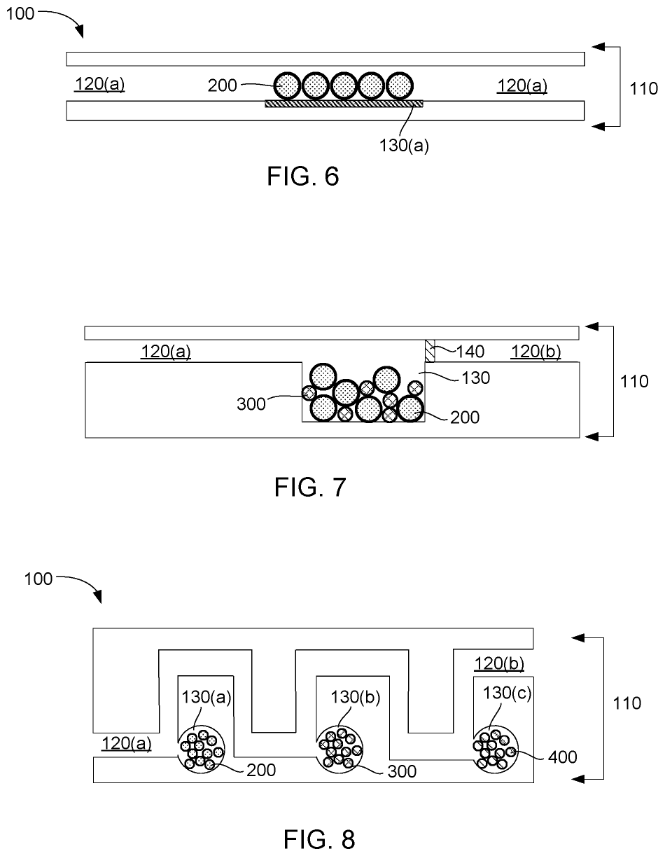

In yet other examples, the microfluidic-retaining region 130 can be in the form of a particle-retaining chemical coating, shown at 130(a) in FIG. 6 that can have an affinity to the degradable polymer 210 or a functional group attached to the degradable polymer of the dry reagent-containing particles 200. For example, the particle-retaining chemical coating can include streptavidin and the degradable polymer can include biotin. In another example, the degradable polymer can include streptavidin and the degradable polymer can include avidin. Streptavidin forms a non-covalent bond with biotin and avidin. In yet another example, the degradable polymer can include alkyne functionalized polylactic acid and/or the particle-retaining chemical coating can include azide functionalized polylactic acid. These functionalized groups can undergo copper(I)-catalyzed azide-alkyne cycloaddition, forming a covalent bond, for example. The particle-retaining chemical coating in some examples can be bound to a microfluidic channel wall surface of the microfluidic-retaining region as depicted in FIG. 6. In another example, the particle-retaining chemical coating can be bound to a microfluidic discontinuity feature such as a wall of a microfluidic cavity, a wall of a microfluidic weir, an exterior surface of the baleen or an a wall of a microfluidic post, a porous membrane, or any combinations thereof.

In some examples, the microfluidic device 100 can include a series of microfluidic cavities, such as that shown schematically by example in FIG. 8. The series of microfluidic cavities (130(a), 130(b), and 130(c) can be individually loaded with dry reagent-containing particles. The microfluidic cavities can be loaded with the same dry reagent-containing particles 200 or with multiple different types of dry reagent-containing particles. For example, the microfluidic cavities can be loaded with the dry reagent-containing particle, a second dry reagent-containing particle 300, and a third dry reagent-containing particle 400. Loading the microfluidic cavities with different types of dry reagent-containing particles can permit a multi-step reaction.

In yet another example, the microfluidic device 100 can further include a configuration to assist in the release of the reagent 202 from the degradable polymer. For example, the microfluidic device can be transparent to ultra-violet light. In another example, the microfluidic device can include a thermal resistor 170 as shown in FIG. 2 by way of example, but could be used in any of the examples shown or described herein. The thermal resistor, if present, can be associated with the microfluidic-retaining region to apply heat to degrade, erode, etc., the degradable polymer or otherwise release the reagent therefrom. In further detail, the thermal resistor can be positioned to thermally interact with the dry reagent-containing particles 200. The thermal resistor can heat a degradable polymer that can be susceptible to heat thereby assisting in degradation of degradable polymer and the release of the reagent therefrom.

Irrespective of configuration, the microfluidic device 100 can include a dry reagent-containing particle 200 positioned within the microfluidic-retaining region 130 of the device 100. The dry reagent-containing particle can include a dry reagent 202 and a reagent carrier 212, e.g., degradable polymer, salt particle with reagent adsorbed thereon, etc. As depicted in FIGS. 1-13, the reagent carrier can be a degradable polymer, for example. Though a general configuration of the dry reagent-containing particles are shown in many of the

FIGS. relative to the device, it is understood that there are many different types of arrangements where polymer and reagent can be combined for use in the devices shown. For example, the dry reagent-containing particle can be in the form of a polymer-encapsulated reagent, reagent dispersed in a polymer matrix, multi-layered polymer-encapsulated reagent, polymer-encapsulated reagent with the reagent dispersed in a polymer matrix, multi-layered polymer-encapsulated reagent with the reagent dispersed in polymer matrix, polymer-encapsulated reagent with reagent dispersed in a polymer shell of the polymer-encapsulated reagent, etc., and/or combinations thereof. A shape of the dry reagent-containing particle is not particularly limited. In some examples, the dry reagent-containing particle can be spherical as depicted in FIGS. 1, 9, 11, and 12; cube-like as depicted in FIG. 10, rectangular, or can have an irregular shape.

The size of the dry reagent-containing particle 200 can also vary. For example, the dry reagent-containing particle can have a D50 particle size that can range from 750 nm to 10 μm, from 1 μm to 8 μm, or from 1 μm to 5 μm. Individual particle sizes can be outside of these ranges, as the “D50 particle size” is defined as the particle size at which about half of the particles are larger than the D50 particle size and the about half of the other particles are smaller than the D50 particle size, by weight.

As used herein, particle size refers to the value of the diameter of spherical particles or in particles that are not spherical can refer to the longest dimension of that particle. The particle size can be presented as a Gaussian distribution or a Gaussian-like distribution (or normal or normal-like distribution). Gaussian-like distributions are distribution curves that may appear essentially Gaussian in their distribution curve shape, but which can be slightly skewed in one direction or the other (toward the smaller end or toward the larger end of the particle size distribution range). Particle size distribution values are not generally related to Gaussian distribution curves, but in one example of the present disclosure, the dry reagent-containing particle can have a Gaussian distribution, or more typically a Gaussian-like distribution with offset peaks at about D50. In practice, true Gaussian distributions are not typically present, as some skewing can be present, but still, the Gaussian-like distribution can be considered to be essentially referred to as “Gaussian.”

The reagent of the dry reagent-containing particle can vary based on the intended use of the microfluidic device. For example, the reagent can include nucleic acid primers when conducting a chain reaction assay. In yet another example, the reagent can include secondary antibodies when conducting ELISA sandwich assays. In some examples, a liquid reagent can be freeze-dried to obtain the reagent in particulate form. In another example, the dry reagent-containing particle can be encapsulated mixture of reagents, such as master mix used in PCR and including polymerase, magnesium salt, buffer, BSA, and others. Primers can be part of this mixture or encapsulated separately and placed in another retaining region (microcavity), for example. A particulate reagent can have a D50 particle size that can range from 500 nm to 500 μm, from 1 μm to 500 μm, from 25 μm to 250 μm, or from 100 μm to 300 μm.

The degradable polymer as used herein can refer to a polymer that degrades, erodes, or dissolves to release dry reagent upon reaction with a release fluid, heat, light, enzymes, or a combination thereof. In some examples, the degradable polymer can be used to prevent a premature reaction of the reagent. The degradable polymer can be un-inhibitive of the desired reaction between the dry reagent and the sample fluid. In one example, the degradable polymer can be inert with respect to the dry reagent and/or the sample fluid. The degradable polymer can be operable to release a dry reagent within a period of time ranging from one second to five minutes, from five seconds to two minutes, or from 30 seconds to three minutes.

The degradable polymer can have a weight average molecular weight that can range from about 10 kDa to about 500 kDa. In other examples, the degradable polymer can have a weight average molecular weight can range from 50 kDa to 300 kDa, from 25 kDa to 250 kDa, from 15 kDa to 450 kDa, or from 100 kDa to 400 kDa. In some examples, the degradable polymer can be water soluble. The degradable polymer can be selected from polylactic acid, biotinylated polylactic acid, polyvinyl alcohol, biotinylated polyvinyl alcohol, polyethylene glycol, biotinylated polyethylene glycol, polypropylene glycol, biotinylated polypropylene glycol, polytetramethylene glycol, biotinylated polytetramethylene glycol, polycarbolactone, biotinylated polycarbolactone, gelatene, biotinylated gelatene, copolymers thereof, or combinations thereof. In one example, the degradable polymer can include biotin. A biotin containing degradable polymer can be used to adhere the dry reagent-containing polymer to the microfluidic-retaining region of the microfluidic substrate. For example, biotin can form a non-covalent bond to streptavidin coated on a surface.

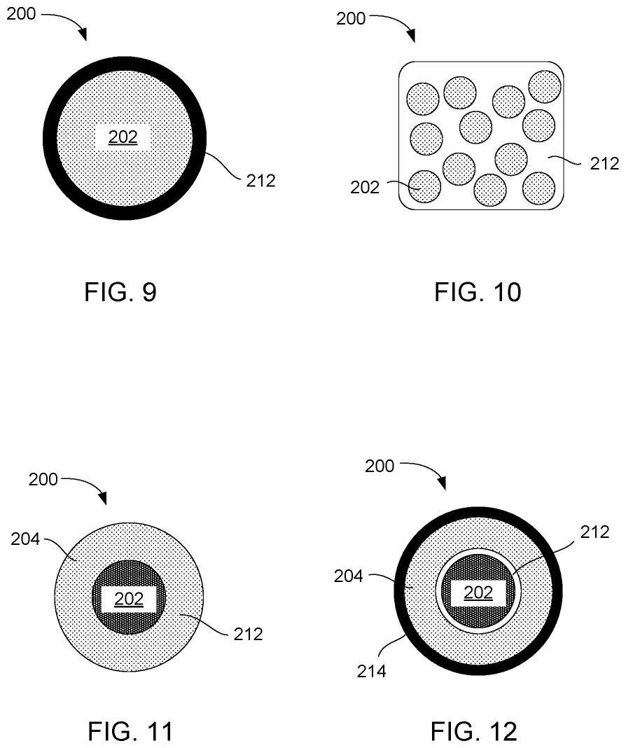

In some examples, the degradable polymer can partially encapsulate or fully encapsulate the reagent to form a dry reagent-containing particle. For example, the reagent carrier 212 can be a degradable polymer can encapsulate the reagent 202 to form a spherical polymer shell and a reagent-containing core as depicted in FIG. 9. The reagent-containing core can include a single reagent particle or can include clumps of reagent.

In one example, the reagent carrier 212 can be polymer and the reagent 202 can be homogenously admixed together and particlized to form particles of polymer matrix with reagent dispersed therein as depicted in FIG. 10. In another example, the dry reagent-containing polymer can include more than one reagent. For example, a degradable polymer shell can further include a second reagent 204. See FIG. 11. In yet another example, the second reagent can be admixed with degradable polymer. The second reagent can coat the degradable polymer and the dry-reagent-containing polymer particle can further include a second reagent carrier 214, which in this case can be a second degradable polymer. See FIG. 12. The second reagent 204 can be different from the reagent 202 of the reagent containing core. The second degradable polymer can be different or the same as the degradable polymer. In still further examples, the dry reagent-containing polymer can include a second degradable polymer that can encapsulate the degradable polymer. The second degradable polymer can be used to control the release of the reagent from the degradable polymer.

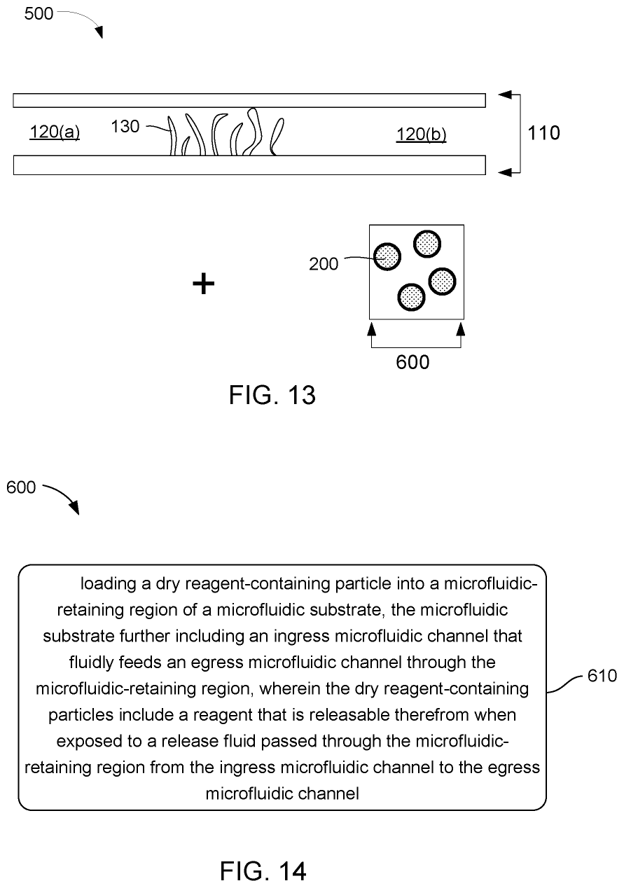

Turning now specifically to the microfluidic system 500. See FIG. 13. The microfluidic system can include a microfluidic substrate 110 that can have an ingress microfluidic channel 120(a) that fluidly feeds an egress microfluidic channel 120(a) through a microfluidic-retaining region 130; dry reagent-containing particles 200; and a fluid carrier 600. The microfluidic-retaining region can include a microfluidic discontinuity feature and/or a particle-retaining chemical coating, for example, as previously described. Though the microfluidic discontinuity in this FIG. shows a microfluidic baleen structure, any of the microfluidic-retaining regions can be used. The dry reagent-containing particles can be combined with the fluid carrier to form a loading fluid used to load the dry reagent-containing particles at the microfluidic discontinuity feature and/or particle-retaining chemical coating. The dry reagent-containing particles can include a reagent that is releasable from the dry reagent-containing particles when exposed to release fluid. The fluid carrier can be combined with the dry reagent-containing particles and can be inert with respect to the dry reagent-containing particles. As used herein, a “fluid carrier” can refer to a fluid that can be used transport the dry reagent-containing particles to the microfluidic-retaining region of the microfluidic substrate. The microfluidic substrate, microfluidic channel, microfluidic-retaining region, and dry reagent-containing particle can be as described previously.

In one example, the system can further include a loading apparatus to load the dry reagent-containing particles carried by a loading fluid of the fluid carrier to a location within the microfluidic-retaining region. The loading apparatus can include a pipette, syringe, dropper, ejector, and the like. The microfluidic discontinuity feature (and/or particle-retaining chemical coating) can be positioned to trap the dry reagent-containing particles at the microfluidic-retaining region. In one example, the loading apparatus can include a fluid ejector. The loading fluid can include a fluid that can be unreactive with respect to the dry reagent-containing particles. In one example, the loading fluid can include a volatile solvent, oil, alcohol, of a combination thereof.

In some examples the system can further include a pump for passing a gas through the microfluidic channel of the microfluidic substrate after passing the fluid carrier there through. The gas can act to remove the loading fluid from the microfluidic channel while retaining the dry reagent-containing particles at the microfluidic-retaining region.

Regardless of the configuration, the microfluidic device and microfluidic system presented herein can be manufactured as part of a microfluidic chip. In one example, the microfluidic chip can be a lab on chip device. The lab on chip device can be a point of care system.

Further presented herein is a method 600 of manufacturing a microfluidic device, as shown in FIG. 14. The method can include loading 610 a dry reagent-containing particle into a microfluidic-retaining region of a microfluidic substrate. The microfluidic substrate can include an ingress microfluidic channel that fluidly feeds an egress microfluidic channel through the microfluidic-retaining region. The dry reagent-containing particles can include a reagent that is releasable therefrom when exposed to a release fluid passed through the microfluidic-retaining region from the ingress microfluidic channel to the egress microfluidic channel. The microfluidic substrate, microfluidic channel, microfluidic-retaining region, discontinuity feature or chemical coating, and dry reagent-containing particle can be as described above.

In one example, the dry reagent-containing particles can include polymer-encapsulated reagent, reagent dispersed in a polymer matrix, multi-layered polymer-encapsulated reagent, polymer-encapsulated reagent with the reagent dispersed in a polymer matrix, multi-layered polymer-encapsulated reagent with the reagent dispersed in polymer matrix, polymer-encapsulated reagent with reagent dispersed in a polymer shell of the polymer-encapsulated reagent, and combinations thereof, wherein when there are multiple reagents or multiple polymers or both, the multiple reagents or multiple polymers or both may be the same or different. In some examples, the reagent can be a liquid phase and freeze-dried within the microfluidic retaining region to form a dry reagent. In yet other examples, the reagent can be loaded as part of a molten polymer/reagent mix.

In one example, loading a dry reagent-containing particle into a microfluidic-retaining region can include loading a reagent into the micro-fluidic-retaining region and laminating the reagent to provide the dry reagent-containing particles within the microfluidic-retaining region. In another example, loading can include passing a loading fluid through the microfluidic-retaining region which includes a fluid carrier and the dry reagent-containing particles and flowing a gas through the microfluidic channel to remove carrier fluid from the microfluidic channel while leaving dry reagent-containing particles at the microfluidic retaining region.

In some examples, the method can further include passing a buffer solution through the microfluidic-retaining region prior to passing the loading fluid therethrough. A buffer solution as used herein can refer to a liquid that is used to maintain a stable pH in a solution. An example of a buffer solution can include phosphate buffered saline, tris buffered saline, or a combination thereof. As an example, a phosphate buffered saline can have a formulation, such as 157 mM Na⁺, 140 mM Cl⁻, 4.45 mM K⁺, 10.1 mM HPO₄²⁻, 1.76 mM H₂PO₄⁻ and a pH of 7.96. This is one specific example and should not be considered to be limiting. Another specific example may be a tris buffered saline having a formulation such as 150 mM NaCl, 50 mM Tris-HCl, pH 7.6.

As used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on presentation in a common group without indications to the contrary.

Concentrations, dimensions, amounts, and other numerical data may be presented herein in a range format. A range format is used merely for convenience and brevity and should be interpreted flexibly to include the numerical values explicitly recited as the limits of the range, and also to include all the individual numerical values or sub-ranges encompassed within that range as if each numerical value and sub-range is explicitly recited. For example, a numeric range that ranges from about 10 to about 500 should be interpreted to include the explicitly recited sub-range of 10 to 500 as well as sub-ranges thereof such as about 50 and 300, as well as sub-ranges such as from 100 to 400, from 150 to 450, from 25 to 250, etc.

The terms, descriptions, and figures used herein are set forth by way of illustration and are not meant as limitations. Many variations are possible within the disclosure, which is intended to be defined by the following claims—and equivalents—in which all terms are meant in the broadest reasonable sense unless otherwise indicated.

What is claimed is:

1. A microfluidic device, comprising:
a microfluidic substrate, including an ingress microfluidic channel that fluidly feeds an egress microfluidic channel through a microfluidic-retaining region, the microfluidic-retaining region including a microfluidic discontinuity feature, a particle-retaining chemical coating, or a combination thereof; and
dry reagent-containing particles including reagent that is releasable from the dry reagent-containing particles when exposed to release fluid, wherein the dry reagent-containing particles are retained within the microfluidic substrate at the microfluidic discontinuity feature or particle-retaining chemical coating in position to release the reagent into the egress microfluidic channel upon flow of release fluid from the ingress microfluidic channel through the microfluidic-retaining region.

2. The microfluidic device of claim 1, wherein the microfluidic discontinuity feature is present and includes a microfluidic cavity, a microfluidic weir, a microfluidic baleen, a microfluidic post, or a combination thereof.

3. The microfluidic device of claim 1, wherein the microfluidic discontinuity feature is present and is associated with a porous membrane positioned downstream therefrom, the porous membrane having an average pore size to permit air, loading fluid, and released reagent in the presence of loading fluid to flow therethrough while prohibiting the dry reagent-containing particles from flowing therethrough while at the size of the particles after loading but before releasing reagent therefrom.

4. The microfluidic device of claim 1, wherein the microfluidic discontinuity is present and comprises a series of microfluidic cavities within the microfluidic-retaining region, wherein the series of microfluidic cavities are individually loaded with the dry reagent-containing particles.

5. The microfluidic device of claim 4, wherein the series of microfluidic cavities are independently loaded with one of multiple different types of dry reagent-containing particles.

6. The microfluidic device of claim 1, wherein the microfluidic discontinuity feature is present and further includes the particle-retaining chemical coating in the form of a streptavidin coating bound to a microfluidic channel wall surface of the microfluidic-retaining region.

7. The microfluidic device of claim 1, wherein the microfluidic discontinuity feature is present and includes a structural feature deviating from the ingress microfluidic channel and the egress microfluidic channel, and wherein the particle-retaining chemical coating is present, wherein the structural feature includes a streptavidin coating bound thereto.

8. The microfluidic device of claim 1, further including a thermal resistor associated with the microfluidic-retaining region and positioned to thermally interact with the dry reagent-containing particles to release reagent therefrom in the presence of a release fluid.

9. A microfluidic system, comprising:
a microfluidic device including:
a microfluidic substrate, including an ingress microfluidic channel that fluidly feeds an egress microfluidic channel through a microfluidic-retaining region, the microfluidic-retaining region including a microfluidic discontinuity feature, a particle-retaining chemical coating, or a combination thereof, and
dry reagent-containing particles including reagent that is releasable from the dry reagent-containing particles when exposed to release fluid; and
a fluid carrier to combine or which is combined with the dry reagent-containing particles that is inert with respect to the dry reagent-containing particles to load the dry-reagent-containing particles at the microfluidic discontinuity feature or the particle-retaining chemical coating.

10. The microfluidic system of claim 9, further comprising a loading apparatus to load the dry reagent-containing particles carried by the loading fluid to a location within the microfluidic-retaining region.

11. The microfluidic system of claim 9, wherein and when loading the dry reagent-containing particles with the loading apparatus, the microfluidic discontinuity or the particle-retaining chemical coating positioned to trap the dry reagent-containing particles at the microfluidic-retaining region.

12. A method of manufacturing a microfluidic device, comprising loading a dry reagent-containing particle into a microfluidic-retaining region of a microfluidic substrate, the microfluidic substrate further including an ingress microfluidic channel that fluidly feeds an egress microfluidic channel through the microfluidic-retaining region, wherein the dry reagent-containing particles include a reagent that is releasable therefrom when exposed to a release fluid passed through the microfluidic-retaining region from the ingress microfluidic channel to the egress microfluidic channel.

13. The method of claim 12, wherein loading a dry reagent-containing particle into a microfluidic-retaining region includes loading a reagent into the micro-fluidic-retaining region and laminating the reagent to provide the dry reagent-containing particles within the microfluidic-retaining region.

14. The method of claim 12, wherein loading includes:
passing a loading fluid through the microfluidic-retaining region which includes a fluid carrier and the dry reagent-containing particles, wherein the fluid carrier is inert with respect to the dry reagent-containing particles; and
flowing a gas through the microfluidic channel to remove carrier fluid from the microfluidic channel while leaving dry reagent-containing particles at the microfluidic retaining region.

15. The method of using the microfluidic device of claim 14, further comprising passing a buffer solution through the microfluidic-retaining region prior to passing the loading fluid therethrough.
